SEQUENCE LISTING (4 sequences)

SEQ ID NO: 1 - DNA, 1936 bp, Aphis gossypii
ccactgcgtcacttccataagtcttgatcatcgtctagccaccaacgacacgacttactgccgtctctgcagcgaaatacgcttccgaataatccgatacagccaaccaccgtcgtgatgaattctaagcccgatggacagcagtccaagtatcagctgtcaaaggatacagctggacttcgttcaacagatttattacctcataatttgtccggacaggcacaaaccagagagtttcttttaaaagtcgttgatatcttagtagattacattgatgacgttaatgatagaaacgaaaaagtattgcattttaagcaccccgaagagatgttacgactgctacaattggatattcccaacgaaggtgtgccattacaaaatttaatcgacgattgcagtctaacactcaagcatcaagtaaaaacaggacatccaagatttttcaaccagctttcatgcggtctagacatcgtgtccatggctggcgaatggctgacggcgacggctaacacgaacatgttcacctacgaaatcgctccagtatttattctcatggaaaacgtggtgttaaccaagatgagagaaatcattgggtggaagaccggcgactcaatttttgctccaggtggatcaatatcgaatatgtacgcgtttttggccgcccgtcataaaatgttcccaggatacaaggaacaaggactccactcgatcaaaggacaactggtcatgtacacatcaaaccaatcgcattattcggttaagagttgtgcatcggtatgcggactaggaaccgaaaattgtgtcgaagtacctagcgacgaaaggggccgcatgataccttctgagctggagcgcctcatattggaaagaaaatccaaaggccacataccgtttttcgtctctgccactgcaggcacgactgttcttggtgcatttgatccaatcaacgacattgcggacatttgcgaaaaatataagctgtggcttcacattgatgctgcctggggtggaggactgcttctatctcgcaagtaccgatatccccgtctggctggcatcgaacgggctaactcagtgacttggaatccacacaaacttatgggcaccctactccagtgctccacaatacattttcgagagaatggaattttgatcagctgcaaccaaatgagcgcggaatacttattcatgcaagacaaactgtacgacgttcaatacgacacaggcgacaaagttatacagtgtggtcgtcacaacgacgtgttcaagctttggcttcaatggcgcgccaagggtaccgaaggtttcgaaaaacacatggatcacttgatggaactcagtgaatatatggtggagaaaattaaagcatcgccagacaaatattatttactccttgaaccggaaatggtgaacgtcagtttttggtacgttccgaagcgcttgcgaaacattccacattctccgaaacgagcggaaagccttggcaagatcacgcctattctgaaggccaaaatgatggaagccggcacgctgatggtagggtatcagccactaaacgagataccgaactttttccggaacattatatccagcgccgcggtcaccaaggaagacgttgactttttgctgtccgaacttgatcgcttgggacaagacctctaaatcaggaggaaaagaaacgattaatgataaatttcgctagtctctataatatatttagttattttattgtgttatgattttgtagacgctatgatcacgattcccggtcaatggctatattcttgccacgcgccgtcaataataataataataataataagtagcctatgctgcgtttataatacagataatcgcataataattaataatttttaattttaaatatatacctatatattataataggtgataagcgttattccaaatcttcctg

SEQ ID NO: 2 - protein, 514 aa, Aphis gossypii
MNSKPDGQQSKYQLSKDTAGLRSTDLLPHNLSGQAQTREFLLKVVDILVDYIDDVNDRNEKVLHFKHPEEMLRLLQLDIPNEGVPLQNLIDDCSLTLKHQVKTGHPRFFNQLSCGLDIVSMAGEWLTATANTNMFTYEIAPVFILMENVVLTKMREIIGWKTGDSIFAPGGSISNMYAFLAARHKMFPGYKEQGLHSIKGQLVMYTSNQSHYSVKSCASVCGLGTENCVEVPSDERGRMIPSELERLILERKSKGHIPFFVSATAGTTVLGAFDPINDIADICEKYKLWLHIDAAWGGGLLLSRKYRYPRLAGIERANSVTWNPHKLMGTLLQCSTIHFRENGILISCNQMSAEYLFMQDKLYDVQYDTGDKVIQCGRHNDVFKLWLQWRAKGTEGFEKHMDHLMELSEYMVEKIKASPDKYYLLLEPEMVNVSFWYVPKRLRNIPHSPKRAESLGKITPILKAKMMEAGTLMVGYQPLNEIPNFFRNIISSAAVTKEDVDFLLSELDRLGQDL

SEQ ID NO: 3 - DNA, 21 bp, Aphis gossypii
ccactgcgtcacttccataag

SEQ ID NO: 4 - DNA, 21 bp, Aphis gossypii
caggaagatttggaataacgc

(19) United States
(12) Patent Application Publication
(10) Pub. No.: US 2007/0077592 A1
(43) Pub. Date: Apr. 5, 2007

(54) Hemipteran glutamate decarboxylase

(76) Inventors: Ruihua Chen, Kendall Park, NJ (US); Susan N. Gilbey, Searsmont, ME (US); Victoria Y. Wong, Enfield, CT (US); Blaik Halling, Newtown, PA (US); Paul Allenza, Flemington, NJ (US)

Correspondence Address: John M. Sheehan;FMC Corporation, 1735 Market Street, Philadelphia, PA, 19103, US

(21) Appl. No.: 10/549,945

(22) PCT Filed: Mar. 19, 2004
(86) PCT No.: PCT/US04/08457

§ 371 Date: Aug. 10, 2006

Related U.S. Application Data
(60) Provisional application No. 60/456,302, filed on Mar. 20, 2003.

(51) Int. Cl. G01N 33/53; C07H 21/04; C12P 21/06; C12N 9/88; A01N 65/00; C12N 5/06

(52) U.S. Cl. 435/7.1; 435/232; 435/69.1; 435/348; 435/320.1; 530/388.26; 536/23.2; 514/2

(57) ABSTRACT

Novel nucleic acid sequences encoding hemipteran L-glutamate decarboxylases, and the amino acid sequence of such protein are disclosed. Methods of making and using the same are disclosed.

### FIELD OF THE INVENTION

The present invention relates to nucleotide sequences that are useful in agrochemical, veterinary or pharmaceutical fields. In particular, the invention relates to nucleotide sequences that encode or may be used to express amino acid sequences that are useful in the identification or development of compounds with (potential) activity as pesticides or as pharmaceuticals. Even more particularly, the invention also relates to the amino acid sequences—such as proteins or polypeptides—that are encoded by, or that may be obtained by suitable expression of, the nucleotide sequences of the invention.

### BACKGROUND OF THE INVENTION

Gamma amino-n-butyric acid (“GABA”) plays an important role in inhibiting synaptic transmission in both vertebrate and invertebrate nervous systems. L-glutamate decarboxylase (“GAD”) is a rate-limiting enzyme involved in the synthesis of GABA. Hence, interruption of GABA synthesis by inhibiting GAD can result in various biological effects (W. Loscher, J. Neurochem., (1981), Vol. 36, No. 4, pp. 1521-1527). As such, there is a desire to develop ways to target this enzyme as a means of identifying biologically active compounds, including insecticides (Gammon et al., Sites of Action of Neurotoxic Pesticides, (1987), Chapter 9, pp. 122-134).

Mammalian GADs, in particular human and mouse GADs, have been cloned and found to be functional when expressed in E. coli and mammalian cells (Huang et al., Proc. Natl. Acad. Sci. U.S.A., (1990), 87(21), pp. 8491-8495; Yamashita et al., Biochem. Biophys. Res. Commun., (1993), 192(3), pp. 1347-52; W. Loscher, J. Neurochem., (1981), Vol. 36, No. 4, pp. 1521-1527; and Davis et al., Biochem. Biophys. Res. Commun., (2000), 267(3), pp. 777-782). Similarly, bacteria, for example, E. coli, Clostridium perfringens, and Lactobacillus brevis, and fungi, for example, Neurospora crassa, GADs have been cloned and expressed (Hao et al., Biochem. J., (1993), 293(3), pp. 735-738; De Biase et al., Biotechnol. Appl. Biochem., (1993), 18(2), pp. 139-142; De Biase et al., Protein Expression Purif., (1996), 8(4), pp. 430438; M. L. Fonda, Methods in Enzymology, (1985), Vol. 113, pp. 11-16; and Ueno et al., (1997), Biosci. Biotech. Biochem., 61 (7), pp. 1168-1171).

In 1979, the Drosophila melanogaster GAD was partial purified (Chude et al., J. Neurochem. (1979), Vol. 32, 1409-1415). Later on, the Drosophila melanogaster GAD was cloned and found to be functional when expressed in oocytes and in mammalian cells (Jackson et al., J. Neurochem., (1990), 54(3), 1068-78; and Phillips et al., J. Neurochem., (1993), 61(4), 1291-301).

### SUMMARY OF THE INVENTION

The present invention relates to novel hemipteran decarboxylase protein, fragments thereof, nucleic acid molecules encoding the novel hemipteran decarboxylase proteinand fragments thereof, antibodies that specifically bind to the novel hemipteran decarboxylase protein, methods of using the novel hemipteran decarboxylase protein including methods of identifying modulators and inhibitors of the same, and methods of inhibinting insect populations by inhibiting the novel hemipteran decarboxylase protein.

The present invention relates to nucleotide sequences that encode polypeptides that are useful in the identification or development of compounds with activity as pesticides or as pharmaceuticals. The present invention also relates to polypeptide sequences that are useful in the identification or development of compounds with activity as pesticides or as pharmaceuticals. These nucleotide sequences and polypeptide sequences, will also be referred to herein as “nucleotide sequences of the invention” and “polypeptide sequences of the invention”, respectively.

Another aspect of the invention relates to the use of the nucleotide sequences of the invention, preferably in the form of a suitable genetic construct as described below, in the transformation of host cells or host organisms, for example for the expression of the amino acid sequences of the invention. The invention also relates to host cells or host organisms that have been transformed with the nucleotide sequences of the invention including those that can express the amino acid sequences of the invention.

In still another aspect, the invention relates to methods for the identification and/or development of compounds that can modulate and/or inhibit the biological activity of the amino acid sequences of the invention, in which the above-mentioned nucleotide sequences, amino acid sequences, genetic constructs, host cells or host organisms may be used. Such methods, which will usually be in the form of an assay or screen, will also be further described below.

In a further aspect the invention relates to methods of controlling insect populations by inhibiting activity or expression of their glutamate decarboxylase protein Such methods, which will usually be in the form of an assay or screen, will also be further described below.

### Definitions

Collectively, the nucleic acids of the present invention will be referred to herein as “nucleic acids of the invention”. Also, where appropriate in the context of the further description of the invention below, the terms “nucleotide sequence of the invention” and “nucleic acid of the invention” may be considered essentially equivalent and essentially interchangeable.

Also, for the purposes of the present invention, a nucleic acid is considered to be “(in) essentially isolated (form)”—for example, from its native biological source—when it has been separated from at least one other nucleic acid molecule and sequence with which it is usually associated. Similarly, a polypeptide is considered to be “(in) essentially isolated (form)”—for example, from its native biological source—when it has been effectively separated from other polypeptide molecules with which it is normally assocaited with. In particular, a nucleic acid or polypeptide is considered “essentially isolated” when it has been purified at least 2-fold, in particular at least 10-fold, more in particular at least 100-fold, and up to 1000-fold or more.

### DETAILED DESCRIPTION OF THE INVENTION

The present invention was established from the finding that the amino acid sequences of the invention can be used as (potential) “target(s)” for in vitro or in vivo interaction with chemical compounds and other factors (with the term “target” having its usual meaning in the art, provide for example the definition given in WO 98/06737). Consequently, compounds or factors that have been identified as interacting with the amino acid sequences of the invention (e.g. by the methods as described herein below) may be useful as active agents in the agrochemical, veterinary or pharmaceutical fields.

In one embodiment, the invention relates to a nucleic acid, preferably in (essentially) isolated form, which nucleic acid comprises a nucleotide sequence of the invention, and in particular the nucleotide sequence of SEQ ID NO: 1. The nucleotide sequence of SEQ ID NO: 1 was derived or isolated from the Aphis gossypii organism, in the manner as further described in the Experimental Part below.

Yet another embodiment relates to a double stranded RNA molecule directed against a nucleotide sequence of the invention (one strand of which will usually comprise at least part of a nucleotide sequence of the invention). The invention also relates to genetic constructs that can be used to provide such double stranded RNA molecules (e.g. by suitable expression in a host cell or host organism, or for example in a bacterial strain such as E. coli). For such constructs, reference is made to Maniatis et al., Molecular Cloning, a Laboratory Manual (Cold Spring Harbor Press, 1989).

In a broader sense, the term “nucleotide sequence of the invention” also comprises:
parts or fragments of the nucleotide sequence of SEQ ID NO: 1;
(natural or synthetic) mutants, variants, alleles, analogs, orthologs (herein below collectively referred to as “mutants”) of the nucleotide sequence of SEQ ID NO: 1, as further described below.
parts or fragments of such (natural or synthetic) mutants;
nucleotide fusions of the nucleotide sequence of SEQ ID NO: 1 (or a part or fragment thereof) with at least one further nucleotide sequence;
nucleotide fusions of (natural or synthetic) mutants (or a part or fragment thereof) with at least one further nucleotide sequence;
   in which such mutants, parts, fragments or fusions are preferably as further described below.

Preferably, a nucleotide sequence of the invention will have a length of at least 500 nucleotides, preferably at least 1,000 nucleotides, more preferably at least 2,000 nucleotides; and up to a length of at most 5,500 nucleotides, preferably at most 5,000 nucleotides, more preferably at most, 4,600 nucleotides.

Examples of parts or fragments of the nucleotide sequence of SEQ ID NO: 1; or a part or fragment of a (natural or synthetic) mutant thereof include, but are not limited to, 5′ or 3′ truncated nucleotide sequences, or sequences with an introduced in frame start codon or stop codon. Also, two or more such parts or fragments of one or more nucleotide sequences of the invention may be suitably combined (e.g. ligated in frame) to provide a further nucleotide sequence of the invention.

Preferably, any such parts or fragments will be such that they comprise at least one continuous stretch of at least 100 nucleotides, preferably at least 250 nucleotides, more preferably at least 500 nucleotides, even more preferably more than 1,000 nucleotides, of the nucleotide sequence of SEQ ID NO: 1.

Also, it is expected that—based upon the disclosure herein—the skilled person will be able to identify, derive or isolate natural “mutants” (as mentioned above) of the nucleotide sequence of SEQ ID NO: 1 from (other individuals of) the same species (for example from an individual of a different strain or line, including but not limited to mutant strains or lines). It is also expected that—based upon the disclosure herein—the skilled person will be able to provide or derive synthetic mutants (as defined hereinabove) of the nucleotide sequence of SEQ ID NO: 1.

In one specific embodiment, the mutant is such that it encodes the nucleotide sequence of SEQ ID NO: 1 or a part or fragment thereof.

Preferably, any mutants as described herein will have one or more, and preferably all, of the structural characteristics or conserved features referred to below for the nucleotide sequences of SEQ ID NO: 1.

In particular, any mutants, parts or fragments as described herein may be such that they at least encode the active or catalytic site of the corresponding amino acid sequence of the invention and a binding domain of the corresponding amino acid sequence of the invention.

Also, any mutants, parts or fragments as described herein will preferably have a degree of “sequence identity”, at the nucleotide level, with the nucleotide sequence of SEQ ID NO 1, of at least 75%, preferably at least 80%, more preferably at least 85%, and in particular more than 90%, and up to 95% or more.

Also, preferably, any mutants, parts or fragments of the nucleotide sequence of the invention will be such that they encode an amino acid sequence which has a degree of “sequence identity”, at the amino acid level, with the amino acid sequence of SEQ ID NO: 2, of at least 50%, preferably at least 60%, more preferably at least 70%, even more preferably at least 80%, and in particular more than 90% and up to 95% or more, in which the percentage of “sequence identity” is calculated as described below.

For this purpose, the percentage of “sequence identity” between a given nucleotide sequence and the nucleotide sequence of SEQ ID NO: 1 may be calculated by dividing the number of nucleotides in the given nucleotide sequence that are identical to the nucleotide at the corresponding position in the nucleotide sequence of SEQ ID NO: 1 by the total number of nucleotides in the given nucleotide sequence and multiplying by 100%, in which each deletion, insertion, substitution or addition of a nucleotide—compared to the sequence of SEQ ID NO:1—is considered as a difference at a single nucleotide position.

Also, in a preferred aspect, any mutants, parts or fragments as described herein will encode proteins or polypeptides having biological activity that is essentially similar to the biological activity described above for the sequences of SEQ ID NO: 1, i.e. to a degree of at least 50%, preferably at least 75%, and up to 90%, as measured by standard assay techniques as described below.

Any mutants, parts or fragments as described herein are preferably such that they are capable of hybridizing with the nucleotide sequence of SEQ ID NO: 1, i.e. under conditions of “moderate stringency”, and preferably under conditions of “high stringency”. Such conditions will be clear to the skilled person, for example from the standard handbooks, such as Sambrook et al. and Ausubel et al., mentioned above, as well as in EP 0 967 284, EP 1085 089 or WO 00/55318.

It is also within the scope of the invention to use a fusion of a nucleotide sequence of the invention (as described above) with one or more further nucleotide sequence(s), including but not limited to one or more coding sequences, non-coding sequences or regulatory sequences. Preferably, in such fusions, the one or more further nucleotide sequences are operably connected (as described below) to the nucleotide sequence of the invention (for example so that, when the further nucleotide sequence is a coding sequence, the nucleotide fusion encodes a protein fusion as described below).

In another embodiment, the invention relates to an antisense molecule against a nucleotide sequence of the invention.

The nucleic acids of the invention may also be in the form of a genetic construct, again as further described below. Genetic constructs of the invention will generally comprise at least one nucleotide sequence of the invention, optionally linked to one or more elements of genetic constructs known per se, as described below. Such genetic constructs may be DNA or RNA, and are preferably double-stranded DNA. The constructs may also be in a form suitable for transformation of the intended host cell or host organism, in a form suitable for integration into the genomic DNA of the intended host cell or in a form suitable independent replication, maintenance and inheritance in the intended host organism. For instance, the genetic construct may be in the form of a vector, such as for example a plasmid, cosmid, a yeast artificial chromosome (“YAC”), a viral vector or transposon. In particular, the vector may be an expression vector, i.e. a vector that can provide for expression in vitro or in vivo (e.g. in a suitable host cell or host organism as described below). An expression vector comprising a nucleotide sequence of the invention is also referred to herein as a recombinant expression vector. These constructs will also be referred to herein as “genetic constructs of the invention”.

In a preferred embodiment, such a construct a recombinant expression vector which will comprise:
a) the nucleotide sequence of the invention; operably connected to:
b) one or more regulatory elements, such as a promoter and optionally a suitable terminator;
   and optionally also:
c) one or more further elements of genetic constructs known per se; in which the terms “regulatory element”, “promoter”, “terminator”, “further elements” and “operably connected” have the meanings indicated herein below.

As the one or more “further elements” referred to above, the genetic construct(s) of the invention may generally contain one or more suitable regulatory elements (such as a suitable promoter(s), enhancer(s), or terminator(s)), 3′- or 5′-untranslated region(s) (“UTR”) sequences, leader sequences, selection markers, expression markers or reporter genes, or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and the transformation technique to be used.

Preferably, in the genetic constructs of the invention, the one or more further elements are “operably linked” to the nucleotide sequence(s) of the invention or to each other, by which is generally meant that they are in a functional relationship with each other. For instance, a promoter is considered “operably linked” to a coding sequence if said promoter is able to initiate or otherwise control or regulate the transcription or the expression of a coding sequence (in which said coding sequence should be understood as being “under the control of” said promoter)

Generally, when two nucleotide sequences are operably linked, they will be in the same orientation and usually also in the same reading frame. They will usually also be essentially contiguous, although this may also not be required.

Preferably, the optional further elements of the genetic construct(s) used in the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling or regulating the transcription or the expression of a nucleotide sequence—e.g. a coding sequence—to which it is operably linked (as defined above).

Such a promoter may be a constitutive promoter or an inducible promoter, and may also be such that it (only) provides for expression in a specific stage of development of the host cell or host organism, or such that it (only) provides for expression in a specific cell, tissue, organ or part of a multicellular host organism.

Some particularly preferred promoters include, but are not limited to, constitutive promoters, such as cytomegalovirus (“CMV”), Rous sarcoma virus (“RSV”), simian virus-40 (“SV40”), for example, pSVL SV40 Late Promoter Expression Vector (Pharmacia Biotech Inc., Piscataway, N.J.), or herpes simplex virus (“HSV”) for expression in mammalian cells or insect constitutive promoters such a the immediate early baculovirus promoter described by Jarvis et al. Methods in Molecular Biology Vol. 39 Baculovirus Expression Protocols ed. C. Richardson. Hamana Press Inc., Totowa, N.J. 1995 available in pIE vectors from Novagen (Novagen, Inc. Madison, Wis.) or insect inducible promoters such as the Drosophila metallothionein promoter described by Bunch et al. Nucleic Acids Research, Vol. 6, No. 3 1043-106, 1988 available in vectors from Invitrogen (Invitrogen Corporation, Carlsbad, Calif.).

Another embodiment of the invention relates to a host cell or host organism that has been transformed or contains a nucleotide sequence, with a nucleic acid or with a genetic construct of the invention. The invention also relates to a host cell or host organism that expresses, or (at least) is capable of expressing (e.g. under suitable conditions), an amino acid sequence of the invention. Collectively, such host cells or host organisms will also be referred to herein as “host cells or host organisms of the invention”.

The host cell may be any suitable (fungal, prokaryotic or eukaryotic) cell or cell line, for example:
a bacterial strain, including but not limited to strains of E. coli, Bacillus, Streptomyces and Pseudomonas;
a fungal cell, including but not limited to cells from species of Aspergillus and Trichoderma;
a yeast cell, including but not limited to cells from species of Kluyveromyces or Saccharomyces;
an amphibian cell or cell line, such as Xenopus oocytes.

In one specific embodiment, which may be particularly useful when the nucleotide sequences of the invention are (to be) used in the discovery and development of insecticidal compounds, the host cell may be an insect-derived cell or cell line, such as:
cells or cell lines derived from Lepidoptera, including but not limited to Spodoptera SF9 and Sf21 cells,
cells or cell lines derived from Aphis;
cells or cell lines derived from Drosophila, such as Schneider and Kc cells; and
cells or cell lines derived from a pest species of interest (as mentioned below), such as from Heliothis virescens.

The host cell may also be a mammalian cell or cell line, including but not limited to CHO- and BHK-cells and human cells or cell lines such as HeK, HeLa and COS.

The host organism may be any suitable multicellular (vertebrate or invertebrate) organism, including but not limited to:
a nematode, including but not limited to nematodes from the genus Caenorhabditis, such as C. elegans,
an insect, including but not limited to species of Aphis, Drosophila, Heliothis, or a specific pest species of interest (such as those mentioned above);
other well known model organisms, such as zebrafish;
a mammal such as a rat or mouse;

Other suitable host cells or host organisms will be clear to the skilled person, for example from the handbooks and patent applications mentioned above.

It should be noted that when a nucleotide sequence of the invention is expressed in a multicellular organism, it may be expressed throughout the entire organism, or only in one or more specific cells, tissues, organs or parts thereof, for example by expression under the control of a promoter that is specific for said cell(s), tissue(s), organ(s) or part(s).

The nucleotide sequence may also be expressed during only a specific stage of development or life cycle of the host cell or host organism, again for example by expression under the control of a promoter that is specific for said stage of development or life cycle. Also, as already mentioned above, said expression may be constitutive, transient or inducible.

Preferably, these host cells or host organisms are such that they express, or are (at least) capable of expressing (e.g. under suitable conditions), an amino acid sequence of the invention (and in case of a host organism: in at least one cell, part, tissue or organ thereof). The invention also includes further generations, progeny and offspring of the host cell or host organism of the invention, which may for instance be obtained by cell division or by sexual or asexual reproduction.

In yet another aspect, the invention relates to a nucleic acid, preferably in (essentially) isolated form, which nucleic acid encodes or can be used to express an amino acid sequence of the invention (as defined herein), and in particular the amino acid sequence of SEQ ID NO: 2.

The amino acid sequence of SEQ ID NO: 2 may be isolated from the species mentioned above, using any technique(s) for protein isolation and purification known to one skilled in the art. Alternatively, the amino acid sequence of SEQ ID NO: 2 may be obtained by suitable expression of a suitable nucleotide sequence—such as the nucleotide sequence of SEQ ID NO: 1 or a suitable mutant thereof—in an appropriate host cell or host organism, as further described below.

In another aspect, the invention relates to a protein or polypeptide, preferably in (essentially) isolated form, said protein or polypeptide comprising an amino acid sequence of the invention (as defined above), in particular the amino acid sequence of SEQ ID NO: 2.

In a broader sense, the term “amino acid sequence of the invention” also comprises:
parts or fragments of the amino acid sequence of SEQ ID NO: 2;
(natural or synthetic) mutants, variants, alleles, analogs, orthologs (herein below collectively referred to as “analogs”) of the amino acid sequence of SEQ ID NO: 2;
parts or fragments of such analogs;
fusions of the amino acid sequence of SEQ ID NO: 2 (or a part or fragment thereof) with at least one further amino acid residue or sequence;
fusions of the amino acid sequence of an analog (or a part or fragment thereof) with at least one further amino acid residue or sequence;
   in which such mutants, parts, fragments or fusions are preferably as further described below.

The term “amino acid sequence of the invention” also comprises “immature” forms of the abovementioned amino acid sequences, such as a pre-, pro- or prepro-forms or fusions with suitable leader sequences. Also, the amino acid sequences of the invention may have been subjected to post-translational processing or be suitably glycosylated, depending upon the host cell or host organism used to express or produce said amino acid sequence; or may be otherwise modified (e.g. by chemical techniques known per se in the art).

Examples of parts or fragments of the amino acid sequence of SEQ ID NO: 2, or a part or fragment of a (natural or synthetic) analog thereof mutant thereof include, but are not limited to, N- and C-truncated amino acid sequence. Also, two or more parts or fragments of one or more amino acid sequences of the invention may be suitably combined to provide an amino acid sequence of the invention.

Preferably, an amino acid sequence of the invention has a length of at least 100 amino acids, preferably at least 250 amino acids, more preferably at least 500 amino acids; and up to a length of at most 2,000 amino acids, preferably at most 1,000 amino acids, more preferably at most 750 amino acids.

Preferably, any such parts or fragments will be such that they comprise at least one continuous stretch of at least 5 amino acids, preferably at least 10 amino acids, more preferably at least 20 amino acids, even more preferably more than 30 amino acids, of the amino acid sequence of SEQ ID NO: 2.

In particular, any parts or fragments as described herein are such that they (at least) comprise the active or catalytic site of the corresponding amino acid sequence of the invention or a binding domain of the corresponding amino acid sequence of the invention. As will be clear to the skilled person, such parts or fragments may find particular use in assay- and screening techniques (as generally described below) and (when said part or fragment is provided in crystalline form) in X-ray crystallography.

Also, it is expected that—based upon the disclosure herein—the skilled person will be able to identify, derive or isolate natural “analogs” (as mentioned above) of the amino acid sequence of SEQ ID NO: 2. Such mutants could be derived from (other individuals of) the same species (for example from an individual of a different strain or line, including but not limited to mutant strains or lines); or from (individuals of) other species. For example, such analogs could be derived from the insect species mentioned above.

It is also expected that—based upon the disclosure herein—the skilled person will be able to provide or derive synthetic “analogs” (as mentioned above) of the amino sequence of SEQ ID NO: 2.

Preferably, any mutants as described herein will have one or more, and preferably all, of the structural characteristics or conserved features referred to below for the sequences of SEQ ID NO: 2.

Preferably, any analogs, parts or fragments as described herein will be such that they have a degree of “sequence identity”, at the amino acid level, with the amino acid sequence of SEQ ID NO: 2 of at least 50%, preferably at least 60%, more preferably at least 70%, even more preferably at least 80%, and in particular more than 90% and up to 95% or more.

For this purpose, the percentage of “sequence identity” between a given amino acid sequence and the amino acid sequence of SEQ ID NO: 2 may be calculated by dividing the number of amino acid residues in the given amino acid sequence that are identical to the amino acid residue at the corresponding position in the amino acid sequence of SEQ ID NO: 2 by the total number of amino acid residues in the given amino acid sequence and multiplying by 100%, in which each deletion, insertion, substitution or addition of an amino acid residue—compared to the sequence of SEQ ID NO: 2—is considered as a difference at a single amino acid (position).

Alternatively, the degree of sequence identity may be calculated using a known computer program, such as those mentioned above.

Also, such sequence identity at the amino acid level may take into account so-called “conservative amino acid substitutions”, which are well known in the art, for example from GB-A-2 357 768, WO 98/49185, WO 00/46383 and WO 01/09300; and (preferred) types or combinations of such substitutions may be selected on the basis of the pertinent teachings from the references mentioned in WO 98/49185.

Also, preferably, any analogs, parts or fragments as described herein will have a biological activity that is essentially similar to the biological activity described above for the sequences of SEQ ID NO: 2, i.e. to a degree of at least 10%, preferably at least 50% more preferably at least 75%, and up to 90%, as measured by standard assay techniques as described below.

It is also within the scope of the invention to use a fusion of an amino acid sequence of the invention (as described above) with one or more further amino acid sequences, for example to provide a protein fusion. Generally, such fusions may be obtained by suitable expression of a suitable nucleotide sequence of the invention—such as a suitable fusion of a nucleotide sequence of the invention with one or more further coding sequences—in an appropriate host cell or host organism, as further described below.

One particular embodiment, such fusions may comprise an amino acid sequence of the invention fused with a reporter protein such as glutathione S-transferase (“GST”), green fluorescent protein (“GFP”), luciferase or another fluorescent protein moiety. As will be clear to the skilled person, such fusions may find particular use in expression analysis and similar methodologies.

In another embodiment, the fusion partner may be an amino acid sequence or residue that may be used in purification of the expressed amino acid sequence, for example using affinity techniques directed against said sequence or residue. Thereafter, said sequence or residue may be removed (e.g. by chemical or enzymatical cleavage) to provide the nucleotide sequence of the invention (for this purpose, the sequence or residue may optionally be linked to the amino acid sequence of the invention via a cleavable linker sequence). Some preferred, but non-limiting examples of such residues are multiple histidine residues and glutatione residues.

In one preferred, but non-limiting aspect, any such fusion will have a biological activity that is essentially similar to the biological activity described above for the sequences of SEQ ID NO: 2, i.e. to a degree of at least 10%, preferably at least 50% more preferably at least 75%, and up to 90%, as measured by standard assay techniques as described below.

The nucleotide sequences and amino acid sequences of the invention may generally be characterized by the presence of one or more of the following structural characteristics or conserved features:

For the gene Aphis gossypii: SEQ ID NO: 1 is a cDNA sequence encompassing the open reading frame; and SEQ ID NO: 2 is the protein encoded by SEQ ID NO: 1.

By analogy to other GADs, it is likely that the functional protein is monomeric. See, e.g., Hannan and Hall, In Comparative Molecular Neurobiology, Y. Pichon, 1993, Birkhuaser Verlag Basel Switzerland).

On the basis of the above, and although the invention is not specifically limited to any specific explanation or mechanism, the nucleotide sequences and amino acid sequences have (biological) activity as a decarboxylase. In particular, the present invention has shown activity as a decarboxylase from insects of the order Hemiptera, which are aphids, leafhoppers, whiteflies, scales and true bugs that have mouthparts adapted to piercing and sucking.

As is known in the art, biological activity of this kind can be measured using standard assay techniques (see I. Cozzani, Analytical Biochem., (1970), 33, pp. 125-131; Scriven et al., Analytical Biochem., (1988), 170, pp. 367-371; Holdiness ea al., Analytical Letters, (1980), 13 (B15), pp. 1333-1344; Heerze et al., Analytical Biochem., (1990), 185, pp. 201-205; G. Zhang and A. W. Bown, Phytochemistry, (1997), Vol. 44, No. 6, pp. 1007-1009; O. Chude and J. Wu, J. Neurochem., (1976), Vol. 27, pp. 83-86; Torchinskiy et al., Doklady Akademii nauk SSR, (1972), Vol. 205, No. 3; and Rosenberg et al., Analytical Biochem., (1989), 181, pp. 59-65).

Another embodiment of the invention relates to a nucleic acid probe that is capable of hybridizing with a nucleotide sequence of the invention under conditions of moderate stringency, preferably under conditions of high stringency, and in particular under stringent conditions (all as described above). Such nucleotide probes may for instance be used for detecting or isolating a nucleotide sequence of the invention or as a primer for amplifying a nucleotide sequence of the invention; all using techniques known per se, for which reference is again made to the general handbooks such as Sambrook et al. and Ausubel et al., mentioned above.

Preferably, when to be used for detecting or isolating another nucleotide sequence of the invention, such a nucleotide probe will usually have a length of between 15 and 100 nucleotides, and preferably between 20 and 80 nucleotides. When used as a primer for amplification, such a nucleotide probe will have a length of between 25 and 75 nucleotides, and preferably between 20 and 40 nucleotides.

Generally, such probes can be designed by the skilled person starting from a nucleotide sequence or amino acid sequence of the invention—and in particular the sequence of SEQ ID NO: 1 or SEQ ID NO: 2—optionally using a suitable computer algorithm.

In a further aspect, the invention relates to methods for preparing mutants and genetic constructs of the nucleotide sequences of the present invention.

Natural mutants of the nucleotide sequences of the present invention may be obtained in a manner essentially analogous to the method described in the Experimental Part, or alternatively by:
construction of a DNA library from the species of interest in an appropriate expression vector system, followed by direct expression of the mutant sequence;
construction of a DNA library from the species of interest in an appropriate expression vector system, followed by screening of said library with a probe of the invention (as described below) or with a nucleotide sequence of the invention;
isolation of mRNA that encodes the mutant sequence from the species of interest, followed by cDNA synthesis using reverse transcriptase;
   or by any other suitable method(s) or technique(s) known per se, for which reference is for instance made to the standard handbooks, such as Sambrook et al., “Molecular Cloning: A Laboratory Manual” (2nd.ed.), Vols. 1-3, Cold Spring Harbor Laboratory Press (1989) and F. Ausubel et al., “Current protocols in molecular biology”, Green Publishing and Wiley Interscience, New York (1987).

Techniques for generating such synthetic sequences of the nucleotide sequences of the present invention will be clear to the skilled person and may for instance include, but are not limited to, automated DNA synthesis; site-directed mutagenesis; combining two or more parts of one or more naturally occurring sequences, introduction of mutations that lead to the expression of a truncated expression product; introduction of one or more restriction sites (e.g. to create cassettes or regions that may easily be digested or ligated using suitable restriction enzymes), and the introduction of mutations by means of a PCR reaction using one or more “mismatched” primers, using for example a sequence of a naturally occurring GAD as a template. These and other techniques will be clear to the skilled person, and reference is again made to the standard handbooks, such as Sambrook et al. and Ausubel et al., mentioned above.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors include:
vectors for expression in mammalian cells: pSVL SV40 (Pharmacia), pMAMneo (Clontech), pcDNA3 (Invitrogen), pMC1neo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565);
vectors for expression in bacterials cells: pET vectors (Novagen) and pQE vectors (Qiagen);
vectors for expression in yeast or other fungal cells: pYES2 (Invitrogen) and Pichia expression vectors (Invitrogen);

vectors for expression in insect cells: pBlueBacII (Invitrogen), pEI1 (Novagen), pMT/V5His (Invitrogen).

In a further aspect, the invention relates to methods for transforming a host cell or a host organism with a nucleotide sequence, with a nucleic acid or with a genetic construct of the invention. The invention also relates to the use of a nucleotide sequence, of a nucleic acid or of a genetic construct of the invention transforming a host cell or a host organism.

According to one specific embodiment, the expression of a nucleotide sequence of the invention in a host cell or host organism may be reduced, compared to the original (e.g. native) host cell or host organism. This may for instance be achieved in a transient manner using antisense or RNA-interference techniques well known in the art, or in a constitutive manner using random, site specific or chemical mutagenesis of the nucleotide sequence of the invention.

Suitable transformation techniques will be clear to the skilled person and may depend on the intended host cell or host organism and the genetic construct to be used. Some preferred, but non-limiting examples of suitable techniques include ballistic transformation, (micro-)injection, transfection (e.g. using suitable transposons), electroporation and lipofection. For these and other suitable techniques, reference is again made to the handbooks and patent applications mentioned above.

After transformation, a step for detecting and selecting those host cells or host organisms that have been successfully transformed with the nucleotide sequence or genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

In yet another aspect, the invention relates to methods for producing an amino acid sequence of the invention.

To produce or obtain expression of the amino acid sequences of the invention, a transformed host cell or transformed host organism may generally be kept, maintained or cultured under conditions such that the (desired) amino acid sequence of the invention is expressed or produced. Suitable conditions will be clear to the skilled person and will usually depend upon the host cell or host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell or host organism used. Also, the amino acid sequence of the invention may be glycosylated, again depending on the host cell or host organism used.

The amino acid sequences of the invention may then be isolated from the host cell or host organism or from the medium in which said host cell or host organism was cultivated, using protein isolation and purification techniques known per se, such as (preparative) chromatography and electrophoresis techniques, differential precipitation techniques, affinity techniques (e.g. using a specific, cleavable amino acid sequence fused with the amino acid sequence of the invention) and preparative immunological techniques (i.e. using antibodies against the amino acid sequence to be isolated).

In one embodiment, the amino acid sequence thus obtained may also be used to generate antibodies specifically against said sequence or an antigenic part or epitope thereof.

In one embodiment, the present invention relates to antibodies, for example monoclonal and polyclonal antibodies, that are generated specifically against amino acid sequences of the present invention, preferably SEQ ID NO: 2, or an analog, variant, allele, ortholog, part, fragment or epitope thereof.

Such antibodies, which form a further aspect of the invention, may be generated in a manner known per se, for example as described in GB-A-2 357 768, U.S. Pat. No. 5,693,492, WO 95/32734, WO 96/23882, WO 98/02456, WO 98/41633 and WO 98/49306. Often, but not exclusively, such methods will involve as immunizing a immunocompetent host with the pertinent amino acid sequence of the invention or an immungenic part thereof (such as a specific epitope), in amount(s) and according to a regimen such that antibodies against said amino acid sequence are raised, and than harvesting the antibodies thus generated, e.g. from blood or serum derived from said host.

For instance, polyclonal antibodies can be obtained by immunizing a suitable host such as a goat, rabbit, sheep, rat, pig or mouse with (an epitope of) an amino acid sequence of the invention, optionally with the use of an immunogenic carrier (such as bovine serum albumin or keyhole limpet hemocyanin) or an adjuvant such as Freund's, saponin, aluminium hydroxide or a similar mineral gel, or keyhole limpet hemocyanin or a similar surface active substance. After a suitable immune response has been raised (usually within 1-7 days), the antibodies can be isolated from blood or serum taken from the immunized animal in a manner known per se, which optionally may involve a step of screening for an antibody with desired properties (i.e. specificity) using known immunoassay techniques, for which reference is again made to for instance WO 96/23882.

Monoclonal antibodies may for example be produced using continuous cell lines in culture, including hybridoma-based and similar techniques, again essentially as described in the above cited references. Accordingly, cells and cell lines that produce monoclonal antibodies against an amino acid sequence of the invention form a further aspect of the invention, as do methods for producing antibodies against amino acid sequences of the invention, which methods may generally involve cultivating such a cell and isolating the antibodies from the culture or medium, again using techniques known per se.

Also, Fab-fragments against the amino acid sequences of the invention (such as F(ab)₂, Fab′ and Fab fragments) may be obtained by digestion of an antibody with pepsin or another protease, reducing disulfide-linkages and treatment with papain and a reducing agent, respectively. Fab-expression libraries may for instance be obtained by the method of Huse et al., 1989, Science 245:1275-1281.

In another embodiment, the amino acid sequence of the invention, or a host cell or host organism that expresses such an amino acid sequence, may also be used to identify or develop compounds or other factors that can modulate the (biological) activity of, or that can otherwise interact with, the amino acid sequences of the invention, and such uses form further aspects of the invention. As will be clear to the skilled person, in this context, the amino acid sequence of the invention will serve as a target for interaction with such a compound or factor.

In this context, the terms “modulate”, “modulation, “modulator” and “target” will have their usual meaning in the art, for which reference is inter alia made to the definitions given in WO 98/06737. Generally, a modulator is a compound or factor that can enhance, inhibit or reduce or otherwise alter, influence or affect (collectively referred to as “modulation”) a functional property of a biological activity or process (for example, the biological activity of an amino acid sequence of the invention).

In this context, the amino acid sequence of the invention may serve as a target for modulation in vitro (e.g. as part of an assay or screen) or for modulation in vivo (e.g. for modulation by a compound or factor that is known to modulate the target, which compound or factor may for example be used as an active compound for agrochemical, veterinary or pharmaceutical use).

For example, the amino acid sequences, host cells or host organisms of the invention may be used as part of an assay or screen that may be used to identify or develop modulators of the amino acid sequence of the invention, such as a primary screen (e.g. a screen used to identify modulators of the target from a set or library of test chemicals with unknown activity with respect to the target) or a secondary assay (e.g. an assay used for validating hits from a primary screen or used in optimizing hit molecules, e.g. as part of hits-to-leads chemistry).

For instance, such an assay or screen may be configured as an in vitro assay or screen, which will generally involve binding of the compound or factor to be tested as a potential modulator for the target (herein below also referred to as “test chemical”) to the target, upon which a signal generated by said binding is measured. Suitable techniques for such in vitro screening will be clear to the skilled person, and are for example described in Eldefrawi et al., (1987). FASEB J., Vol. 1, pages 262-271 and Rauh et al., (1990), Trends in Pharmacol. Sci., vol. 11, pages 325-329. For example, such an assay or screen may be configured as a binding assay or screen, in which the test chemical is used to displace a detectable ligand from the target (e.g. a radioactive or fluorescent ligand), upon which the amount of ligand displaced from the target by the modulator is determined.

Such an assay or screen may also be configured as a cell-based assay or screen, in which a host cell of the invention is contacted with or exposed to a test chemical, upon which at least one biological response by the host cell is measured.

Also, such an assay or screen may also be configured as an whole animal screen, in which a host organism of the invention is contacted with or exposed to a test chemical, upon which at least one biological response (such as a phenotypical, behavioral or physiological change, including but not limited to paralysis or death) by the host organism is measured.

Thus, generally, the assays and screens described above will comprise at least one step in which the test chemical is contacted with the target (or with a host cell or host organism that expresses the target), and in particular in such a way that a signal is generated that is representative for the modulation of the target by the test chemical. In a further step, said signal may then be detected.

Accordingly, in one aspect, the invention relates to a method for generating a signal that is representative for the interaction of an amino acid sequence of the invention with a test chemical, said method at least comprising the steps of:
a) contacting the amino acid sequence of the invention, or a host cell or host organism containing or expressing an amino acid sequence, with said test chemical, in such a way that a signal may be generated that is representative for the interaction between said test chemical and said amino acid sequence; and optionally
b) detecting the signal that may thus be generated.

In another aspect, the invention relates to a method for identifying modulators and/or inhibitors of an amino acid sequence of the invention (e.g. from a set or library of test chemicals), said method at least comprising the steps of:
a) contacting the amino acid sequence of the invention, or a host cell or host organism containing or expressing an amino acid sequence, with a test chemical, in such a way that a signal may be generated that is representative for the interaction between said test chemical and said the target; and optionally
b) detecting the signal that may thus be generated, said signal identifying the modulator and/or inhibitor of said amino acid sequence.

Accordingly, the present invention provides methods of identifying a modulator and/or inhibitor of a hemipteran GAD protein activity. In preferred embodiments, the hemipteran GAD protein used in the methods has an amino acid sequence selected from the group consisting of SEQ ID NO: 2, a mutant thereof, and a fragment thereof. In some embodiments, the nucleic acid sequence that encodes the hemipteran GAD is SEQ ID NO: 1.

A test chemical may be part of a set or library of compounds, which may be a diverse set or library or a focussed set or library, as will be clear to the skilled person. The libraries that may be used for such screening can be prepared using combinatorial chemical processes known in the art or conventional means for chemical synthesis.

The assays and screens of the invention may be carried out at medium throughput to high throughput, for example in an automated fashion using suitable robotics. In particular, in this embodiment, the method of the invention may be carried out by contacting the target with the test compound in a well of a multi-well plate, such as a standard 24, 96, 384, 1536 or 3456 well plate.

Usually, in a screen or assay of the invention, for each measurement, the target or host cell or host organism will be contacted with only a single test compound. However, it is also within the scope of the invention to contact the target with two or more test compounds—either simultaneously or sequentially—for example to determine whether said combination provides a synergistic effect.

Once a test chemical has been identified as a modulator and/or inhibitor for an amino acid sequence of the invention (e.g. by means of a screen or assay as described hereinabove), it may be used per se as a modulator and/or inhibitor of the relevant amino acid sequence of the invention, preferably, an amino acid sequence of SEQ ID NO: 2, a mutant thereof, and a fragment thereof, more preferably SEQ ID NO: 2 (e.g. as an active substance for agrochemical, veterinary or pharmaceutical use), or it may optionally be further optimized for final use, e.g. to improve properties such as solubility, adsorption, bio-availability, toxicity, stability, persistence, environmental impact, etc. It will be clear to the skilled person that the nucleotide sequences, preferably SEQ ID NO: 1, amino acid sequences, host cells or host organisms and methods of the invention may find further use in such optimization methodology, for example as (part of) secondary assays.

The invention is not particularly limited to any specific manner or mechanism in or via which the modulator and/or inhibitor (e.g. the test chemical, compound or factor) modulates, inhibits, or interacts with, the target (in vivo or in vitro). For example, the modulator and/or inhibitor may be a competitive inhibitor, a non-competitive inhibitor, a cofactor, an allosteric inhibitor or other allosteric factor for the target, or may be a compound or factor that enhances or reduces binding of target to another biological component associated with its (biological) activity, such as another protein or polypeptide, a receptor, or a part of organelle of a cell. As such, the modulator and/or inhibitor may bind with the target (at the active site, at an allosteric site, at a binding domain or at another site on the target, e.g. covalently or via hydrogen bonding), block and/or inhibit the active site of the target (in a reversible, irreversible or competitive manner), block and/or inhibit a binding domain of the target (in a reversible, irreversible or competitive manner), or influence or change the conformation of the target.

As such, the test chemical, modulator and/or inhibitor may for instance be:
an analog of a known substrate of the target;
an oligopeptide, e.g. comprising between 2 and 20, preferably between 3 and 15 amino acid residues;
an antisense or double stranded RNA molecule;
a protein, polypeptide;
a cofactor or an analog of a cofactor.

The test chemical, modulator and/or inhibitor may also be a reference compound or factor, which may be a compound that is known to modulate, inhibit or otherwise interact with the target (e.g. a known substrate or inhibitor for the target) or a compound or factor that is generally known to modulate, inhibit or otherwise interact with other members from the general class to which the target belongs (e.g. a known substrate or inhibitor of said class).

Preferably, however, the test chemical, modulator and/or inhibitor is a small molecule, by which is meant a molecular entity with a molecular weight of less than 1,500, preferably less than 1,000. This may for example be an organic, inorganic or organometallic molecule, which may also be in the form or a suitable salt, such as a water-soluble salt. The term “small molecule” also covers complexes, chelates and similar molecular entities, as long as their (total) molecular weight is in the range indicated above.

As already mentioned above, the compounds or factors that have been identified or developed as modulators and/or inhibitors of the amino acid sequences of the invention, preferably, an amino acid sequence of SEQ ID NO: 2, a mutant thereof, and a fragment thereof, more preferably SEQ ID NO: 2, (and precursors for such compounds) may be useful as active substances in the agrochemical, veterinary or pharmaceutical fields, for example in the preparation of agrochemical, veterinary or pharmaceutical compositions, and both such modulators as well as compositions containing them further aspects of the invention.

For example, in the agrochemical field, the modulators and/or inhibitors of the invention may be used as an insecticide, nematicide, molluscide, helminticide, acaricide or other types of pesticides or biocides, e.g. to prevent or control (infestations with) harmful organisms, both as contact agents and as systemic agents. As such, the modulators and/or inhibitors may for example be used as a crop protection agent, as a pesticide for household use, or as an agent to prevent or treat damage caused by harmful organisms (e.g. for the protection of seed, wood or stored crops or fruits). Preferably, the modulators and/or inhibitors of the invention are used as insecticides.

For any such application, one or more modulators and/or inhibitors of the invention may be suitably combined with one or more agronomically acceptable carriers, adjuvants or diluents—and optionally also with one or more further compounds known per se with activity as (for example) a plant protection agent (to broaden the spectrum of action and optionally to provide a synergistic effect), herbicide, fertilizer or plant growth regulator—to provide a formulation suitable for the intended final use. Such a formulation may for example be in the form of a solution, emulsion, dispersion, concentrate, aerosol, spray, powder, flowable, dust, granule, pellet, fumigation candle, bait or other suitable solid, semi-solid or liquid formulation, and may optionally also contain suitable solvents, emulsifiers, stabilizers, surfactants, antifoam agents, wetting agents, spreading agents, sticking agents, attractants or (for a bait) food components. Reference is made to the standard manuals, such as “Pesticidal Formulation Research”, ACS-publications (1969) and “Pesticide Formulations”, Wade van Valkenburg Ed, Marcel Dekker publications (1973).

Such compositions may generally contain one or more modulators and/or inhibitors of the invention in a suitable amount, which generally may be between 0.1 and 99%, and in particular between 10 and 50%, by weight of the total composition.

The modulators and/or inhibitors and compositions of the invention may be particularly useful as insecticides, for example to combat or control undesired or harmful insects (both adult and immature forms, such as larvae) from following orders:
Coleoptera, such as Pissodes strobi, Diabrotica undecimpunctata howardi, and Leptinotarsa decemlineata;
Diptera, such as Rhagoletis pomonoella, Mayetiola destructor, and Liriomyza huidobrensis;
Hymenoptera, such as Neodiprion taedae tsugae, Camponotus pennsylvanicus, and Solenopsis wagneri;
Hemiptera, such as Pseudatomoscelis seriatus, Lygus lineolaris (Palisot de Beauvois), Acrosternum hilare, and Aphis gossypii
Homoptera; and
Lepidoptera such as Heliothis virescens.

When used to control harmful or undesired organisms, these organisms may be directly contacted with the modulators, inhibitors, or compositions of the invention in an amount suitable to control (e.g. kill or paralyze) the organism. This amount may be readily determined by the skilled person (e.g. by testing the compound on the species to be controlled) and will usually be in the region of between particular between 10 and 500 g/ha, in particular between 100 and 250 g/ha.

The modulators, inhibitors, or compositions of the invention may also be applied systemically (e.g. to the habitat of the organism to be controlled or to the soil), and may also be applied to the plant, seed, fruit etc. to be protected, again in suitable amounts, which can be determined by the skilled person. The modulators and/or inhibitors of the invention may also be incorporated—e.g. as additives—in other compositions known per se, for example to replace other pesticidal compounds normally used in such compositions.

In one specific embodiment, the modulators and/or inhibitors and compositions of the invention may be used in the fields of agrochemical, veterinary or human health to prevent or treat infection or damage or discomfort caused by parasitic organisms, and in particular by parasitic arthropods, nematodes and helminths such as:
ectoparasitic arthropods such as ticks, mites, fleas, lice, stable flies, horn flies, blowflies and other biting or sucking ectoparasites;
endoparasites organisms such as helminths;
   and also to prevent or treat diseases that are caused or transferred by such parasites. For such purposes, the modulators and/or inhibitors of the invention may for example be formulated as a tablet, an oral solution or emulsion, an injectable solution or emulsion, a lotion, an aerosol, a spray, a powder, a dip or a concentrate.

In the fields of animal and human health, the modulators, inhibitors, and compositions of the invention may also be used for the prevention or treatment of diseases or disorders in which the amino acid sequence of the invention may be involved as a target. For this purpose, the modulators and/or inhibitors of the invention may be formulated with one or more additives, carriers or diluents acceptable for pharmaceutical or veterinary use, which will be clear to the skilled person.

Thus, in a further aspect, the invention relates to the use of a modulator and/or inhibitor of the invention in the preparation of a composition for agrochemical, veterinary or pharmaceutical use, as described hereinabove. The invention relates to the use of the modulators, inhibitors and compositions of the invention in controlling harmful organisms and in preventing infestation or damage caused by harmful organisms, again as described above.

The invention will now be further illustrated by means of the following non-limiting Experimental Part.

Experimental Part:

### EXAMPLE 1

### Cloning of Cotton Aphid (“CA”) Glutamate Decarboxylase

1. Isolation of poly(A⁺) RNA.

Cotton aphids were collected from cotton plants and placed in ice-chilled glass centrifuge tubes which had been cleaned and baked for 6 hours at 180° C. prior to use. Aliquots of approximately 0.4 gram of cotton aphids was used for isolation of poly(A⁺) RNA.

Diethyl pyrocarbonate (DEPC)-treated water was made by incubating DEPC (Aldrich Chemical Co., Inc. Milwaukee, Wis.) in water at concentration of 0.1% (v/v) for 16 hours at room temperature, followed by autoclaving. The microprobe of a Braun homogenizer (B. Brawn Biotech International, Allentown, Pa.) was soaked in 100% ethanol and dried prior to use.

RNA isolation was done using QuickPrep mRNA Purification kit (Amersham Pharmacia biotech, Piscataway, N.J.) according to the manufacturer's instruction. All the buffers and solutions mentioned here are included in the kit. An aliquot of 0.4 gram of cotton aphid was homogenized at full speed in 1.5 ml chilled extraction buffer until it is in a uniform suspension. After adding 3 ml of elution buffer, the sample was homogenized again briefly and the resulting mixture was centrifuged at approximately 12000×g for 10 minutes at room temperature. The supernatant was used for poly(A⁺) RNA isolation. After application of supernatant to the resin of oligo(dT)-cellulose spun column, washing with high salt and low salt buffers, the bound poly(A⁺) RNA was eluted with three washes of 0.25 ml elution buffer pre-warmed to 65° C. To precipitate the mRNA, 50 μl of K Acetate solution, 10 μl of Glycogen solution, and 1 ml of 95% Ethanol were added to 0.5 ml of elute. The mixture was placed at −20° C. for one hour and then centrifuged at maximal speed at room temperature in an eppendorf microcentrifuge. Precipitated poly(A⁺) RNA was then dissolved in 50 μl DEPC-treated water and stored at −80° C. until use.

2. Reverse Transcription and PCR Amplification (RT-PCR).

Bioinformatics research indicates that two of the ESTs (Expressed Sequence Tag), both of which from FMC proprietary Aphis gossypii EST (Expressed Sequencing Tag) library, are the partial transcripts of our target gene. These two ESTs have extensive coverage on both 5′ end and 3′ end, including the 5′ UTR and 3′ UTR. The cloning strategy was to select gene-specific sense primer from known 5′ UTR, and gene-specific antisense pimer from known 3′ UTR. The sense primer was CCACTGCGTCACTTCCATAAG, and the antisense primer was CAGGAAGATTTGGAATAACGC.

RT-PCR was done using the Titanium™ One-Step RT-PCR kit. RT-PCR Master Mix (43.5 μl per reaction) was prepared according to the manufacturer's protocol. RT-PCR reaction was run at the volume of 50 μl containing the following components: 0.5 μl each of primers (45 μM), 2 μl of poly(A⁺) RNA (0.2 μg/μl), and 3.5 μl of DEPC-treated water. RT-PCR was run on a Perkin Elmer cycler using the following conditions: 50° C. for 60 min, 94° C. for 5 min, followed 40 cycles of the PCR reaction: 94° C. for 30 sec, 65° C. for 30 sec, and 68° C. for 60 sec. The completion of cycling was followed by incubation at 68° C. for 2 min.

3. Subcloning of RT-PCR Product and Sequencing.

From above-described RT-PCR reaction we obtained very small quantity of PCR product which is not sufficient for sequencing. We have tried re-amplification by eLONGase and sub-cloning by restriction digestion into cloning vectors but failed. We then use pCRII-TOPO vector (Invitrogen, Carlsbad, Calif.) for sub-cloning. We directly used PCR product from Taq amplification in TOPO cloning; or before TOPO cloning, we incubated PCR product from eLONGase amplification with Taq polymerase at 72° C. to add A's to the PCR product. TOPO cloning was done according to manufacturer's instruction. The resulting plasmid was sequencd using T7 or SP6 as sequencing primers.

Primers. The primers utilized were as follows:

| | | | |
|---|---|---|---|
| Primer | Sequence | Translation | Orientation |

| | | | |
|---|---|---|---|
| 1 | CCACTGCGTCACTTCCATAAG | N/A | Forward |
| 2 | CAGGAAGATTTGGAATAACGC | N/A | Reverse |

1. A substantially pure protein having the amino acid sequence selected from the group consisting of: SEQ ID NO: 2, a mutant thereof, and a fragment thereof.

2. The protein of claim 1 wherein said protein has the amino acid sequence of SEQ ID NO: 2.

3. The protein of claim 1 wherein said protein has the amino acid sequence of a fragment of SEQ ID NO: 2.

4. An isolated nucleic acid molecule that comprises a nucleic acid sequence that encodes the protein of claim 1.

5. A recombinant expression vector comprising the nucleic acid molecule of claim 4.

6. A host cell comprising the recombinant expression vector of claim 5.

7. An isolated nucleic acid molecule that comprises a nucleic acid sequence that encodes the protein of claim 2.

8. A recombinant expression vector comprising the nucleic acid molecule of claim 7.

9. A host cell comprising the recombinant expression vector of claim 8.

10. An isolated nucleic acid molecule having a nucleic acid sequence selected from the group consisting of: SEQ ID NO: 1 and a fragment thereof having at least 10 nucleotides.

11. The isolated nucleic acid molecule of claim 10, wherein the nucleic acid sequence is SEQ ID NO: 1.

12. A recombinant expression vector comprising the nucleic acid molecule of claim 11.

13. A host cell comprising the recombinant expression vector of claim 12.

14. An isolated antibody which binds to an epitope on SEQ ID NO: 2.

15. A method of identifying a modulator of a Hemipteran glutamate decarboxylase protein activity comprising the steps of:
contacting the amino acid sequence of the invention, or a host cell or host organism containing or expressing an amino acid sequence, with a test chemical, in such a way that a signal may be generated that is representative for the interaction between said test chemical and said target; and optionally
detecting the signal that may thus be generated, said signal identifying the modulator of said amino acid sequence.

16. The method of claim 15 wherein said Hemipteran glutamate decarboxylase protein has an amino acid sequence selected from the group consisting of: SEQ ID NO: 2, a mutant thereof, and a fragment thereof.

17. The method of claim 16 wherein said Hemipteran glutamate decarboxylase protein has an amino acid sequence of SEQ ID NO: 2.

18. A method of identifying an inhibitor of a Hemipteran glutamate decarboxylase protein activity comprising the steps of:
contacting the amino acid sequence of the invention, or a host cell or host organism containing or expressing an amino acid sequence, with a test chemical, in such a way that a signal may be generated that is representative for the interaction between said test chemical and said target; and optionally
detecting the signal that may thus be generated, said signal identifying the inhibitor of said amino acid sequence.

19. The method of claim 18 wherein said Hemipteran glutamate decarboxylase protein has an amino acid sequence selected from the group consisting of: SEQ ID NO: 2, a mutant thereof, and a fragment thereof.

20. The method of claim 19 wherein said Hemipteran glutamate decarboxylase protein has an amino acid is SEQ ID NO: 2.

21. A method of preparing an isolated protein having the amino acid sequence selected from the group consisting of: SEQ ID NO:2, a mutant thereof, and a fragment thereof comprising the step of isolating said protein from a host cell of claim 6.

22. A method of controlling an insect, comprising contacting the insect with the modulator of glutamate decarboxylase.

23. The method of claim 22 wherein the insect is a Hemipteran insect and the glutamate decarboxylase is a Hemipteran glutamate decarboxylase.

24. The method of claim 23 wherein the is Hemipteran glutamate decarboxylase has an amino acid sequence comprising SEQ ID NO:2.

25. The method of claim 23 wherein the is Hemipteran glutamate decarboxylase is encoded by a nucleic acid sequence comprising SEQ ID NO:1.
